(19) United States
(12) Patent Application Publication
(10) Pub. No.: US 2026/0315663 A1
(43) Pub. Date: Oct. 15, 2026

(54) Biodegradable, Self-Retentive and Anti-Reflux Intra-Ureteral Stent

(71) Applicants: Fundación Centro de Cirugia de Mínima Invasión Jesus Uson, Caceres (ES); Fundación para la Investigación Biomédica del Hospital Universitario Ramón y Cajal (FIBIO-HRC), Madrid (ES); Instituto Murciano de Investigación y desarrollo agrario y medioambiental (IMIDA), La Alberca (ES)

(72) Inventors: Federico SORIA GÁLVEZ, Cáceres (ES); Salvador David AZNAR CERVANTES, Murica (ES)

(73) Assignees: Fundación Centro de Cirugia de Mínima Invasión Jesus Uson, Caceres (ES); Fundación para la Investigación Biomédica del Hospital Universitario Ramón y Cajal (FIBIO-HRC), Madrid (ES); Instituto Murciano de Investigación y desarrollo agrario y medioambiental (IMIDA), La Alberca (ES)

(21) Appl. No.: 19/485,367

(22) PCT Filed: May 19, 2023
(86) PCT No.: PCT/IB2023/000230

§ 371 Date: Nov. 18, 2025

(51) Int. Cl. A61L 31/04; A61F 2/04; A61L 31/10; A61L 31/14; A61L 31/16

(52) U.S. Cl. CPC A61L 31/043; A61F 2/04; A61L 31/10; A61L 31/148; A61L 31/16; A61L 2300/402; A61L 2300/406; A61L 2300/41; A61L 2300/416

(57) ABSTRACT

Intra-ureteral stent that comprises: a) a jay shaped hollow tube (1) with biomechanical memory on its proximal end, which serves to insert and anchor the device in the ureter; b) a main body (2) made of biodegradable intertwined threads (4) which facilitate urine drainage and; c) a distal end (3), configured to remain inside of the ureter; wherein the intertwined threads (4) are biodegradable and in that the main body (2) is coated with a polymeric matrix (5), the polymeric matrix (5) comprising at least one active ingredient.

### TECHNICAL FIELD OF THE INVENTION

The present invention belongs to the field of implantable medical devices. Particularly, the present invention relates to the field of ureteral stents, more particularly to an intra-ureteral stents which does not cause vesicoureteral reflux (anti-reflux) is biodegradable and locally provides an active ingredient.

### BACKGROUND OF THE INVENTION

The first ureteral catheter was created near the beginning of 1900 by Joaquin Albarran. In 1967, Zimskind was responsible for a significant step forward in the research and development of ureteral catheters. In his publication, the author describes the long-term use of silicon ureteral catheters inserted by cystoscopy. These catheters moved around easily because they were not anchored at either end. To reduce the movement, Gibbons designed a silicon catheter with several anchoring points along its length. However, these anchoring points significantly increased the outer diameter, were difficult to insert, and reduced the range of urinary flow.

The next design was aimed at preventing anterograde or retrograde displacement by creating an angle and forming a jay shape on both ends. In 1978, the authors Finney and Hepperlan described the double jay ureteral catheter. Later, to improve the double jay catheter, Mardis developed the double pigtail catheter, which had a lower tendency to move.

It is important to differentiate between ureteral catheters and ureteral stents, though both devices can be called stents. Ureteral catheters are usually plastic prostheses which go from the renal pelvis to the bladder (double jay or double pigtail catheters); whereas ureteral stents are usually made of metal, similar to vascular stents, and have the sole purpose of dilating a short section of the ureter in the manner of a bypass.

By definition, a ureteral catheter is a device made preferably from synthetic polymer biomaterial designed to drain urine and remain between the renal pelvis and the bladder. The urinary system is an unstable environment chemically, with a supersaturation of mucous and crystalloid substances, which in the long term creates a significant problem in relation to the biocompatibility and bio durability of the catheter.

The ideal catheter should be designed to last, chemically stable, made from a biocompatible polymer, resistant to incrustation and infection, resist ureteral peristalsis, and move as little as possible. The catheter should ideally be easy to insert and comfortable for the patient, reacting minimally with the tissue. It should be highly elastic and enable the increase of urine flow from the moment it is inserted. In addition, it is preferably radiopaque and easily removed by endoscope or alternatively not require removal.

Another indispensable property of ureteral catheters is biomechanical memory to return to the dimensions it had before being physically distorted. This property is needed so that the catheter can be inserted using a guide, and then return to its jay shape configuration after the guide is removed. This memory allows the catheter to be retained without significant displacement. At present, all authors say that the ideal ureteral catheter does not exist, though there are a great variety of devices.

Double jay or pigtail catheters have become an integral part of contemporary urologic practice. These catheters prevent or alleviate ureteral obstructions due to a wide range of intrinsic or extrinsic causes. These include: ureteral lithiasis, ureteral stenosis, congenital anomalies such as obstruction of the ureteropelvic junction, fibrosis or retroperitoneal tumours, neoplasms which extrinsically affect the upper ureter, or iatrogenic injuries. They are also widely used to provide urinary drainage following surgery of the upper ureter, independently of the procedure, or after an endourologic (ureteroscopy, lithiasis, endopyelotomy, endoureterotomy, etc.) or laparoscopic (pyeloplasty, ureteroneocystostomy, etc.) procedure, or by conventional surgery, as well as to dilate the upper ureter.

Payne and Ramsay observed that draining the bladder with a ureteral catheter caused a significant increase in intrarenal pressure, suggesting that vesicoureteral reflux contributes to increasing said pressure.

The ureter is then dilated after ureteral intubation. The ureteral catheter is used to therapeutically increase the ureteral diameter to examine the lumen and the passive exit of ureteral stones. This dilation can serve as a protective mechanism to minimise intrapelvic pressure resulting from the mechanical obstruction caused by the catheter.

The ureter is dilated due to the presence of the ureteral catheter because there is a reduction in peristalsis (physiological process consisting of radially symmetric contractions and relaxations which push urine from the ureter to the bladder), which is a result of the catheters impeding the coaptation of ureter walls. Mostly found a reduction of peristalsis in 80% of patients with a ureteral catheter. For this reason, the physiological flow of the ureter with a ureteral catheter would probably drain as a result of hydrostatic forces and gravity. Ureteral dilation and ineffective peristalsis remain for three weeks after the ureteral catheter is removed, meaning the ureterovesical junction is also dilated and we will encounter reflux during that time, prolonging the adverse effects of current ureteral catheters after they are removed.

Therefore, another known adverse effect in the ureter is vesicoureteral reflux. When a double jay ureteral catheter is inserted, the anti-reflux valve of the ureterovesical junction is interrupted and the ureteral meatus is kept open by the catheter. This vesicoureteral reflux only reaches the distal ureter, but during the physiological emptying of the bladder, pressure increases, and reflux reaches the kidney. The reflux is produced in 80% of patients with a double jay catheter during emptying, and in 63% during vesical filling.

The range of adverse effects following ureteral catheterisation depends on many factors. These include the catheter material, the length of time it is in place, and presence or lack of an infection. Experimental studies show there is no reduction in renal filtration due to the catheter in the short term, and that there is renal dilation, inflammation changes, and vesicoureteral reflux, though all of these changes are temporary and disappear a few weeks after the ureteral catheter has been removed.

Therefore, it is proven that current commonly used double jay or double pigtail catheters lead to an increase in intrapelvic pressure, hydroureter, vesicoureteral reflux, and overall swelling of the ureteral wall with histological changes in the urothelium. After 1-3 weeks of ureteral catheterisation, the vesical mucus shows severe inflammation and ulceration with occasional metaplasia.

These effects cause significant morbidity in carriers, including: lumbar pain, suprapubic pain, dysuria, haematuria, microhaematuria, urinary infection, nocturia, frequent urination, tenesmus, urgency, incontinence, etc. The aetiology of this high percentage of morbidity associated to double jay ureteral catheters is not well known, but it has been shown that one of the determining factors is the increase of pressure in the renal pelvis due to vesicoureteral reflux caused by ureteral catheters, especially during vesical emptying. Another factor is irritation in the vesical trigone due to the intravesical section of the ureteral catheter. These two factors may be sufficient to explain the symptoms described above, given that the pain is a result of increased pelvic pressure, and the rest are symptoms associated to the irritation and erosion which can be caused by the vesical end of the ureteral catheters.

Therefore, the current conclusion is that in spite of new biomaterials, coatings, and designs which have been tested up to now, the ideal ureteral catheter does not yet exist, and we need to keep working to improve the design of these devices in order to reduce the adverse effects suffered by patients.

Drugs have also been used to reduce the discomfort and adverse effects of ureteral catheters (reduce vesicoureteral reflux), with alpha-blockers the muscles of the intramural part of the ureter, vesical trigone, and prostate such as alfuzosin. However, in spite of improvements, there is no reduction in the consumption of analgesics.

There is therefore as need for improved ureteral stents that can further reduce the adverse effects associated to these medical devices, avoid the need for replacements, and facilitate local treatments that can serve as coadjutant or additional treatments for ureteral and ureteral-related medical problems.

### SUMMARY OF THE INVENTION

A first aspect of the present invention refers to an intra-ureteral stent. The intra-ureteral stent comprises:
a) a jay shaped hollow tube with biomechanical memory on its proximal end, which serves to insert and anchor the device in the ureter;
b) a main body made of biodegradable intertwined threads which facilitate urine drainage and;
c) a distal end, configured to remain inside of the ureter.

The intra-ureteral stent is characterised in that the intertwined threads are biodegradable and in that the main body is coated with a polymeric matrix, the polymeric matrix comprising at least one active ingredient.

According to a preferred embodiment, the polymeric matrix comprises fibroin, preferably silk-fibroin which in turn comprises the active ingredient, preferably embedded within the fibroin.

According to another preferred embodiment, the at least one active ingredient is selected from the group consisting of: chemotherapy agents, immunotherapy agents, antibiotic agents, and/or anti-inflammatory agents. More preferably, the chemotherapy agent is selected from any one of the following list: mitomycin, epirubicin, paclitaxel, docetaxel, valrubicin and/or doxorubicin.

According to another preferred embodiment, the polymeric matrix is configured to biodegrade and elute the at least one active ingredient in a controlled pace through hydrolysis.

In another preferred embodiment, the biodegradable intertwined threads are configured to biodegrade in a controlled pace and predictable fashion through hydrolysis.

According to another preferred embodiment, the biodegradable intertwined threads comprise a polymer and/or copolymer. More preferably, the polymer and/or copolymer comprise any of the materials selected from of the following list consisting of: glycomer-631, polyglactin, polycaprolate, polylactic acid, polyglycolic acid, alginate, gellen, poly-4-hydroxybutyrate.

A second aspect of the invention refers to a composition comprising an antitumoral agent for use in the treatment of urothelial cancer, wherein the antitumoral agent is administered by an intra-ureteral stent as any one of the intra-ureteral stents defined in the first aspect of the invention.

The second aspect of the invention also refers to a composition comprising an antitumoral agent for use as an adjuvant therapy in the treatment of urothelial cancer, wherein the composition is administered by an intra-ureteral stent as any one of the intra-ureteral stents 5 defined in the first aspect of the invention.

In a preferred embodiment of any one of the embodiments of the second aspect of the invention, the composition is for use in urothelial cancer wherein the urothelial cancer is urothelial carcinoma. In another preferred embodiment of any one of the embodiments of the second aspect of the invention the composition is for use in urothelial cancer wherein the urothelial cancer is urothelial neoplasm associated to bladder cancer.

The present invention also refers to a composition comprising an analgesic and/or anti-inflammatory agent for use in the treatment of urinary tract inflammations, wherein the analgesic and/or anti-inflammatory agent is administered by an intra-ureteral stent as any one of the intra-ureteral stents defined in the first aspect of the invention.

Lastly, the present invention also refers to a composition comprising an antibiotic agent for use in the treatment of urinary tract infections and/or bacteriuria, wherein the antibiotic agent is administered by an intra-ureteral stent as any one of the intra-ureteral stents defined in the first aspect of the invention.

### BRIEF DESCRIPTION OF THE DRAWINGS

To enable a better understanding of the present disclosure, and to show how the present disclosure may be carried out, reference will now be made, by way of example only, to the accompanying schematic drawings, wherein:

FIG. 1 shows a schematic view of an intra-ureteral stent according to one or more embodiments of the invention.

FIG. 2 shows a close perspective view of the body of an intra-ureteral stent according to one or more embodiments of the invention.

FIG. 3 shows a graphic representation of a nephroureteral unit with an intra-ureteral catheter according to one or more embodiments of the invention correctly placed in the ureter.

FIG. 4 shows a boxplot representation of the concentration of MMC release by an intra-ureteral catheter according to one or more embodiments of the invention.

FIG. 5 shows a boxplot representation of the cell viability assessment (%) of different stents including an intra-ureteral catheter according to one or more embodiments of the invention.

FIG. 6 shows a boxplot representation of the weight loss between the start of a study (T0) and the start of stent degradation (T1) of different stents including an intra-ureteral catheter according to one or more embodiments of the invention.

FIG. 7 shows a boxplot representation of the degradation rate assessment (days) between T1 (onset of degradation); T2 (50% of stent degradation); T3 (complete stent degradation) of different stents including an intra-ureteral catheter according to one or more embodiments of the invention.

FIG. 8 shows a boxplot representation of the pH throughout a study until complete stent degradation of different stents including an intra-ureteral catheter according to one or more embodiments of the invention.

FIG. 9 shows a boxplot representation of the stent thickness at baseline (TO) of different stents including an intra-ureteral catheter according to one or more embodiments of the invention

FIG. 10 shows a graph of the serum creatinine (A), serum urea (B), and Serum GPT (C) levels of an inv-vivo study using an intra-ureteral catheter according to one or more embodiments of the invention.

FIG. 11 shows a graph of the concentration of MMC (mg/mL) released between 0 and 30 h by an intra-ureteral catheter according to one or more embodiments of the invention.

FIG. 12 shows a graph of the pH of the urine of an inv-vivo study using an intra-ureteral catheter according to one or more embodiments of the invention until complete stent degradation.

FIG. 13 shows a boxplot of the hydronephrosis score of an inv-vivo study using an intra-ureteral catheter according to one or more embodiments of the invention until complete stent degradation.

### DESCRIPTION OF THE INVENTION

### Definitions

It must be noted that, as used herein, the singular forms “a”, “an”, and “the”, include plural references unless the context clearly indicates otherwise. Further, unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

It is noted that the term “about”, as used herein, refers to +/−30%, preferably +/−20%, preferably +/−15%, more preferably +/−10%, of the indicated referred value.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. Any of the aforementioned terms (comprising, containing, including, having), whenever used herein in the context of an aspect or embodiment of the present invention may be substituted with the term “consisting of”, though less preferred.

When used herein “consisting of” excludes any element, step, or ingredient not specified in the claim element. When used herein, “consisting essentially of does not exclude materials or steps that do not materially affect the basic and novel characteristics of the claim.

The term “intra-ureteral stent” refers to a ureteral stent that does not reach the vesical trigone.

### DESCRIPTION

Each embodiment disclosed herein is contemplated as being applicable to each of the other disclosed embodiments. Thus, all combinations of the various elements described herein are within the scope of the invention. It should also be understood that, unless clearly indicated to the contrary, in any methods claimed herein that include more than one step or act, the order of the steps or acts of the method is not necessarily limited to the order in which the steps or acts of the method are recited

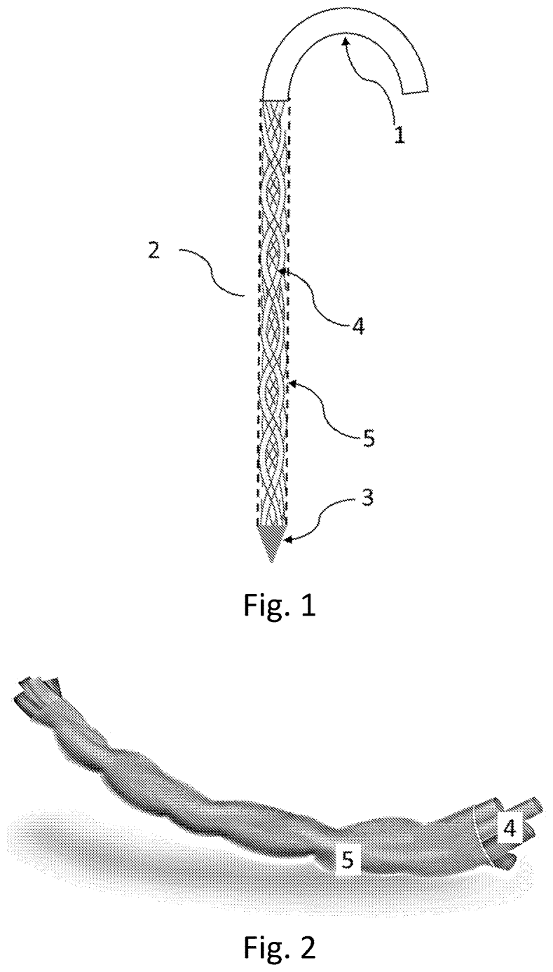

A first aspect of the invention relates to an intraureteral stent. As shown with respect to FIG. 1his new ureteral stent is made up of 3 main elements which make up a single element:
a. a jay shaped hollow tube (1) with biomechanical memory on its proximal end, which serves to insert and anchor the device in the ureter;
b. a main body (2) made of biodegradable intertwined threads (4) which facilitate urine drainage and;
c. a distal end (3), the distal end configured to remain of the ureter.

The hollow tube (1) is for introducing and fixing the device. It is radiopaque and jay shaped so it can be easily detected. It is placed in the renal pelvis to fix the catheter, making it self-retentive. This element has biomechanical memory, which makes it easier to insert, and once the insertion guide wire has been removed, it returns to its native jay shape, stopping the catheter from moving. At the same time, its hollow design allows the catheter to be placed under endoscopic or fluoroscopic control by inserting a guide wire along the same axis. It is noted that the anchoring of the stent to the renal pelvis allows the stent to remain in place such that it is not dragged down the ureter at any time.

The main body (2) comprises a series of intertwined threads (4) which have a variable length depending on the patient. This element does not have an internal channel, and urinary drainage takes place in a periprosthetic manner, around the catheter. Thus, there is an increase in the diameter of urinary evacuation in the vesical direction and the use of hollow tubular designs, which increase the external diameter of catheters and can be temporarily blocked by deposits of salts and detritus in urine, is avoided. Furthermore, this element fulfils the supportive purpose in the ureteral healing process, and it allows the passive dilation of the ureteral segment in where it is located.

The intertwined design of the threads, combined with the absence of a metal centre in this segment, increase the flexibility of the catheter, which is good for the ureter.

The intertwined threads (4) are biodegradable with a controlled and predictable biodegradation rate. Advantageously, it decreases the discomfort of patients, reduces the anxiety, the infection rate, prevents bacterial adhesion, reduces health expenses, avoids the “syndrome of forgotten stent” and reduces or avoids the need of anaesthesia in paediatric patients. Also, the intertwined designs of the threads (4) allow for the degradation into smaller segments, which do not obstruct the ureter, which in turn avoids further obstructive issues.

Moreover, as shown in detail in FIG. 2 the main body (2) of the intra-ureteral stent is coated with a polymeric matrix (5). The polymeric matrix (5) comprises at least one active ingredient. It is noted that the polymeric matrix (5) may comprise one or more active ingredients and that the active ingredients may have one or more effects on the urothelial tissue surrounding the stent and/or downstream in the bladder or the urethra. The at least one active ingredient is preferably embedded in the polymeric matrix (5), such that the polymeric matrix (5) comprises embedded within the at least one active ingredient. As the active ingredient is therefore located in the outermost part of the body (2), and the polymeric matrix (5) is conformed to the intertwined threads (4), the active ingredient is provided to the urothelial tissue.

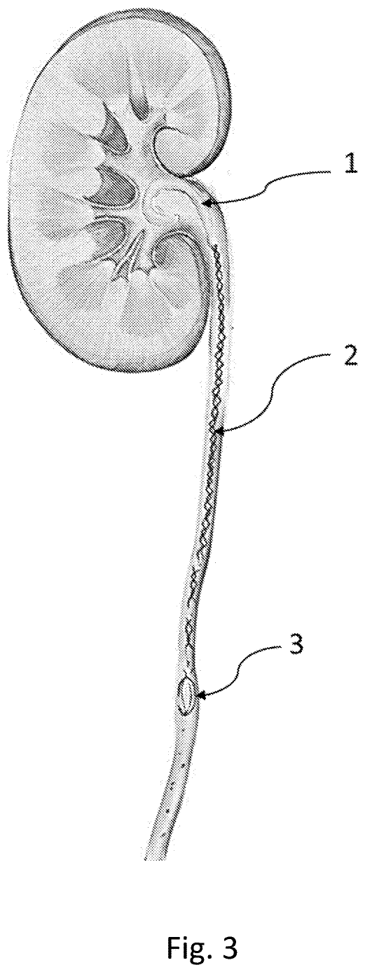

A unique feature of the design of this intra-ureteral stent is that its positioning is exclusively intra-ureteral and at no point goes beyond the ureterovesical junction; this is, this catheter does not have an intravesical section. As such, the catheter does not cause vesicoureteral reflux and, since its distal end does not sit on the vesical neck, it avoids all the complications associated to current ureteral catheters (lumbar pain, suprapubic pain, dysuria, haematuria, microhaematuria, urinary infection, urinary incontinence, nocturia, frequent urination, tenesmus, urgency, incomplete vesical emptying, etc.). Its unique design allows urine to flow normally from the kidney to the bladder, avoiding obstructions which can arise in current catheters. As shown in FIG. 3, once the stent is placed and fixed in the renal pelvis, it becomes fixed to the renal pelvis (R) in a stable manner, and the distal end (3) is configured to remail inside of the ureter.

Also, as the intertwined threads (4) are biodegradable, the body (2) can be eliminated once the purpose of the active ingredient and/or the stent has finalised, therefore providing an intra-ureteral stent that doesn't have to be removed, therefore avoiding the need for a second intervention to extract it.

Therefore, advantageously, the proposed intra-ureteral stent herein provided is specifically designed to not only reduce the problems associated to these types of devices, especially the vesicoureteral reflux, but it further helps providing at least one active ingredient locally into the ureter and is configured to biodegrade once a certain amount of time has gone by. Given the amount of inflammatory and infectious events related to intra-ureteral stents, the proposed intra-ureteral stent can help not only reduce such problems, but even locally treat them or further provide other therapeutic treatments.

It is noted that FIG. 1 shows a schematic drawing of an intra-ureteral stent according to the present invention. However, it comprises many features that could be in different forms in other embodiments of the present invention. For example, the shape of the hollow tube (1) can be more or less curved, as show in FIG. 3, although always respecting its jay-shape when placed in position. Also, the body (2) of FIG. 2 has a particular length, although in other embodiments the body may be longer or shorter. As the body (2) is coated in the polymeric matrix (5) comprising the at least one active ingredient, the length of the body (2) may be adapted depending on the treatment to be administered through the intra-ureteral stent. Also, the distal end (3) is schematically drawn as a triangle, although there are many possible shapes the distal end can take (3).

Likewise, it's noted that FIG. 2 shows a perspective view of the body of an intra-ureteral stent wherein the body (2) comprises 4 intertwined threads (4). However, in other embodiments, the body (2) may comprise more or less intertwined threads (4). FIG. 3 also shows a particular intra-ureteral stent according to the present invention once implanted, that in other embodiments could have different shapes or sizes, such as a different distal end (3).

According to a preferred embodiment of the first aspect of the invention, the polymeric matrix (5) comprises fibroin, preferably silk-fibroin (SF) which in turn comprises the active ingredient, preferably embedded within the fibroin. Advantageously, silk has excellent biocompatibility, high strength, mechanical toughness, robust flexibility, high processability, tuneable degradation, ease of processing, and ease acquisition. The stability of SF coatings can be modified by immersion in methanol, which allows the Beta sheet content to be modified. This is related to the rate of degradation and to the release of the drugs embedded in the SF coating.

According to another preferred embodiment of the first aspect of the invention, the at least one active ingredient is selected from the group consisting of: chemotherapy agents, immunotherapy agents, antibiotic agents and/or anti-inflammatory agents. Advantageously, this allows for the intra-ureteral stent to be able to provide chemotherapeutic, immunotherapeutic, antibiotic and/or anti-inflammatory treatments to the urothelial tissue, including tissue surrounding the stent and/or downstream in the bladder or the urethra. The skilled person may note there is a plurality of these active ingredients that can be provided within the polymeric matrix (5) to provide one or more of such treatments.

In a preferred embodiment, the chemotherapy agent is selected from any of the following list: mitomycin, epirubicin; paclitaxel; docetaxel; valrubicin; and/or doxorubicin. In a particular preferred embodiment, the chemotherapy agent is mitomycin.

According to another preferred embodiment of the first aspect of the invention, the polymeric matrix (5) is configured to biodegrade and elute the at least one active ingredient in a controlled pace through hydrolysis.

Therefore, as urine flows through the ureter, the polymeric matrix (5) biodegrades, and the at least one active ingredient is released into the ureter walls and in the stream, therefore also affecting all the tissues downstream, such as the bladder and the urethra. It is also noted that the polymeric matrix (5) can be designed with different active ingredient concentrations at different depths, such that as the polymeric matrix (5) degrades due to urine hydrolysis, the active ingredient is released at different rates.

Also, the intra-ureteral stent can be configured to release the at least one active ingredient according to a planned treatment. For example, given an active ingredient with chemotherapeutic properties, the polymeric matrix (5) may be comprised of different layers to provide a chemotherapy treatment as a sequence of phases. For example, an outermost layer which will be degraded first comprising a first chemotherapy cycle, followed by an intermediate layer of a week worth of polymeric matrix (5) without active ingredient followed by the innermost layer of the polymeric matrix (5) which will be degraded last comprising a second chemotherapy cycle.

According to another preferred embodiment of the first aspect of the invention, the biodegradable intertwined threads (4) are configured to biodegrade in a controlled pace and predictable fashion through hydrolysis.

Thus, as urine flows through the ureter, the intertwined threads (4) start degrading into the urine through hydrolysis. There are many ways in which the biodegradable intertwined threads (4) can be configured to biodegrade in a controlled pace and predictable fashion through hydrolysis. For example, the threads (4) may be comprised of different materials each with different degradation rates. They may also change the materials depending on the layer, such that the inner of a thread is made of a particular material and the outermost part is made of another material. This allows for different configurations that the skilled person may envisage to make the intertwined threads (4) to biodegrade in a controlled pace and predictable fashion. This allows for the provision of a stent that can be biodegraded once the treatment it was designed for is finished. This treatment may be the stent implantation itself but may also consider the additional treatment the active ingredient is configured to provide.

In a preferred embodiment, different polymers and/or copolymers with different degradation rates are used. Advantageously, because of its braided design and the composition of its polymers, which have different degradation rates, the stent breaks into small fragments that cause no obstructions during their elimination in the urine.

According to another preferred embodiment of the first aspect of the invention, the biodegradable intertwined threads (4) comprise a polymer and/or copolymer. In a preferred embodiment, the polymer and/or copolymer comprise any of the materials selected from the following list consisting of: glycomer-631, polyglactin, polycaprolate, polylactic acid, polyglycolic acid, alginate, gellen, poly-4-hydroxybutyrate.

A second aspect of the invention refers to a composition comprising an antitumoral agent for use in the treatment of urothelial cancer, wherein the composition is administered by the intra-ureteral stent as defined in any one of the embodiments of the first aspect of the invention.

An alternative embodiment of the second aspect of the invention relates to a composition comprising an antitumoral agent for use as an adjuvant therapy in the treatment of urothelial cancer, wherein the composition is administered by the intra-ureteral stent as defined in any one of the embodiments of the first aspect of the invention. Therefore, after the resection of a tumoral tissue, the recurrence can be prevented through the administration of this composition. Advantageously, the local administration of antitumoral agents, helps to reduce the antitumoral dose needed. Also, as the intra-ureteral stent is located in the upper tract, the composition can be used for multiple urothelial carcinoma treatments, including vesicular urothelial carcinomas and upper tract urothelial carcinomas.

In a preferred embodiment of any one of the embodiments of the second aspect of the invention, the composition for use is for use in the treatment of urothelial carcinoma. In another preferred embodiment of any one of the embodiments of the second aspect of the invention, the composition for use is for use in the treatment of urothelial neoplasm associated to bladder cancer and/or urothelial carcinoma associated to the ureter.

Another embodiment of the second aspect of the invention refers to a composition comprising an analgesic and/or anti-inflammatory agent for use in the treatment of urinary tract inflammations, wherein the analgesic and/or anti-inflammatory agent is administered by the intra-ureteral stent as defined in any one of the embodiments of the first aspect of the invention.

Another embodiment of the second aspect of the invention refers to a composition comprising an antibiotic agent for use in the treatment of urinary tract infections and/or bacteriuria, wherein the antibiotic agent is administered by the intra-ureteral stent as defined in any one of the embodiments of the embodiments of the first aspect of the invention.

All of the above are fully within the scope of the present disclosure and are considered to form the basis for alternative embodiments in which one or more combinations of the above-described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

### EXAMPLES

### Example 1: MC Release Study in a Urothelial Carcinoma Cell Culture

### Materials and Methods

### 2.1. BraidStent-SF-MMC

### 2.1.1. Materials for Biodegradable Ureteral Stent Preparation

To perform the in vitro study, 24 fragments of a 10 mm long BraidStent® (JUMISC, Cáceres, Spain) biodegradable ureteral stent were made by combining biodegradable polymers and copolymers-Glycomer™ 631 and polyglycolic acid (PGA), at a ratio of 54% and 46%, respectively. Two 0.17 mm thick threads of Glycomer™ 631 and two 0.14 mm thick threads of PGA, all 10 mm long, were used to manufacture the stent. The threads were then braided together to constitute the central core of the stent. These 24 stent fragments all underwent coating with SF, and 12 of them subsequently underwent the addition of a chemotherapeutic agent widely used for the intracavitary instillation in the upper tract urothelial carcinomas (UTUC), namely mitomycin C (MMC).

### 2.1.2. Materials for Stent SF and MMC Coating

Cocoons of Bombyx mori were obtained from worms reared in the sericulture facilities of the Imida Instituto Murciano de Investigación y Desarrollo Agrario y Ambiental (IMIDA), Biotechnology, Genomics and Plant Breeding Department (La Alberca, Murcia, Spain).

Cocoons were chopped up and boiled in 0.02 M Na₂CO₃ for 30 min to eliminate the sericin. Then, the raw SF was rinsed with distilled water and dried at room temperature for 3 days.

Subsequently, SF was dissolved in 9.3 M LiBr (Acros Organics) for 3 h at 60° C., yielding a 20% weight per volume (w/v) dissolution that was dialysed against distilled water for 3 days (Snakeskin Dialysis Tubing 3.5 KDa MWCO, Thermo Scientific, Rockford, IL, USA), with eight total water changes (at 4° C.). The resultant 7-8% w/v SF solution was recovered and used for the preparation of the coated stents, adjusting the concentration to 7% w/v before use.

The coating of the BraidStent-SF-MMC stent was carried out with powdered 70 mg pure MMC (Mitomycin CRS, Sigma-Aldrich, Darmstadt, Germany). To this end, the concentration employed for both the fibroin, and methanol solutions was 10 mg/mL, and 10 dip-coating cycles were performed by dissolving the drug (10 mg/mL MMC) for 30 min under orbital agitation by stirring at 120 rpm. These two solutions were used to alternately coat the stent fragments by dipping them in the first SF solution for 5 s and then in the methanol solution for 5 s, allowing them to dry for 1 min before repeating the procedure.

The stents were completely stable in room air. However, the MMC coating needs special care (i.e., it must be packaged in lightproof packaging).

2.1.3. Determination and Assessment of the MMC Release from BraidStent-SF-MMC

To determine MMC release, we tested 10 fragments of BraidStent-SF-MMC that were 10 mm in length and coated as described above in artificial urine (AU) (Human Synthetic Urine, BioIVT, West Sussex, UK). The stent fragments were incubated in an orbital shaker incubator under mimicked biological conditions (36.5° C. with 5% CO₂ at 90 rpm). The concentration of MMC released in the AU at 3 and 6 h was determined by HPLC-DAD. Urine from each follow-up was replaced and analysed. The permeability and release kinetics of the MMC depends on the SF coating and is related to the percentage of beta-sheet structure, which is controlled by dipping the SF solution in methanol.

The HPLC-DAD method is an isocratic method with an acetonitrile mobile phase.

Ultrapure water that was 80:20 volume per volume (v/v) at a flow rate of 1 mL/min was used, and separation was performed on a LUNA C18 using 250 mm, 4.6 mm, 5 μm columns at 30° C. The MMC was detected with a diode array detector (DAD) at 365 nm (1260 Infinity II Prime LC System, Agilent Technologies, Santa Clara, CA, USA).

### 2.2. T24 Cell Culture Line

Urothelial carcinoma cells from human bladder cancer T24 cells were used as a model cell line (EP-CL-0227, BioNova científica®, Madrid, Spain) [18,19]. This is a tumour cell line from human transitional bladder cell carcinoma and is frequently used to assess the cellular cytotoxicity of chemotherapeutics in the urinary tract in vitro.

Human T24 cells were seeded in plates containing McCoy's 5a (Thermo Fisher Scientific®, Madrid, Spain) and supplemented with 10% foetal bovine serum (FBS), 1% penicillin/streptomycin, and 1% glutamine and were incubated at 37° C. at 95% relative humidity and 5% CO₂. Cells were maintained at 37° C. in a humidified 5% CO₂ atmosphere for 48 h.

### 2.3. In Vitro Cytotoxicity of the BraidStent-SF-MMC

At 80% confluence, adherent cells were detached with Trypsin EDTA solution (Lonza Bioscience, Pontevedra-Spain) and seeded in four 24-well plates at a final concentration of 20,000 cells per well. Once adherent, four groups of studies were performed. In group 1 (G1), cell viability was assessed after the instillation of pure mitomycin C was added at a concentration of 0.66 mg/mL, the concentration recommended for the adjuvant treatment of UTUC in medical practice [8]. Group 2 (G2), the BraidStent-SF-MMC stent group, is the subject of the current study. The negative controls used were the BraidStent-SF without MMC coating (group 3, G3) and T24 cells in an MMC-free medium (group 4, G4). The sample size was six samples per group. All of these groups were assessed at baseline (T0), at 3 h (T3), and at 6 h (T6).

Mitomycin C cytotoxicity was assessed by a viability assay using CCK-8 (Cell Counting Kit-8, Boster Biological Technology, Pleasanton, CA, USA). The protocol was carried out according to the manufacturer's recommendations, and the absorbance at 450 nm was recorded using a Synergy™ Mx microplate reader (BioTek Instruments, Winooski-Vermont-USA). Briefly, after the first 24 h of incubation, the cell viability assay was performed with CCK-8 at TO in two wells from each experimental group. Next, 10 μL of CCK-8 solution was added to each well of the plates together with Gibco™ DMEM complete FBS (10% FBS, 1% penicillin/streptomycin, 1% glutamine) (ThermoFisher Scientific, Madrid, Spain).

Subsequently, they were kept in the incubator at 37° C. for 45 min. Finally, absorbance was measured using a Synergy™ Mx microplate reader (BioTek Instruments, Winooski-Vermont-USA) at 450 nm. After ending the CCK-8 assay at T0, the study groups were maintained in culture on their respective plates for 3 h and 6 h at 37° C. in the HeraCell™150i CO₂ incubator (Thermo Scientific™, Waltham, MA, USA), with six replicates per experimental group [21]. The absorbance of formazan was measured at 450 nm using the plate reader mentioned above. The percentage (%) of cell viability was calculated as follows:

$$Cell viability ( \% ) = \frac{Absorbance of sample}{Absorbance of control} \times 100 \%$$

### 2.4. Statistical Analysis

Statistical analysis was performed with the SPSS 25.0 program for Windows (IBM, USA). The variable of study was cell viability expressed as the % of cell viability. The normality of the data was analysed using the Shapiro-Wilk test. Student's t-test was used to compare the concentration of MMC released by the BraidStent-SF-MMC at 3 and 6 h. A comparison between groups at 3 and 6 h was carried out using the Kruskal-Wallis test, and, in the case of statistical significance, a corresponding post hoc analysis was carried out using the Bonferroni test. The trend of % of cell viability over time at 3 and 6 h was analysed via the Wilcoxon signed-rank test. The confidence interval set at 95% (95% CI), and significance was determined with p<0.05.

### Results

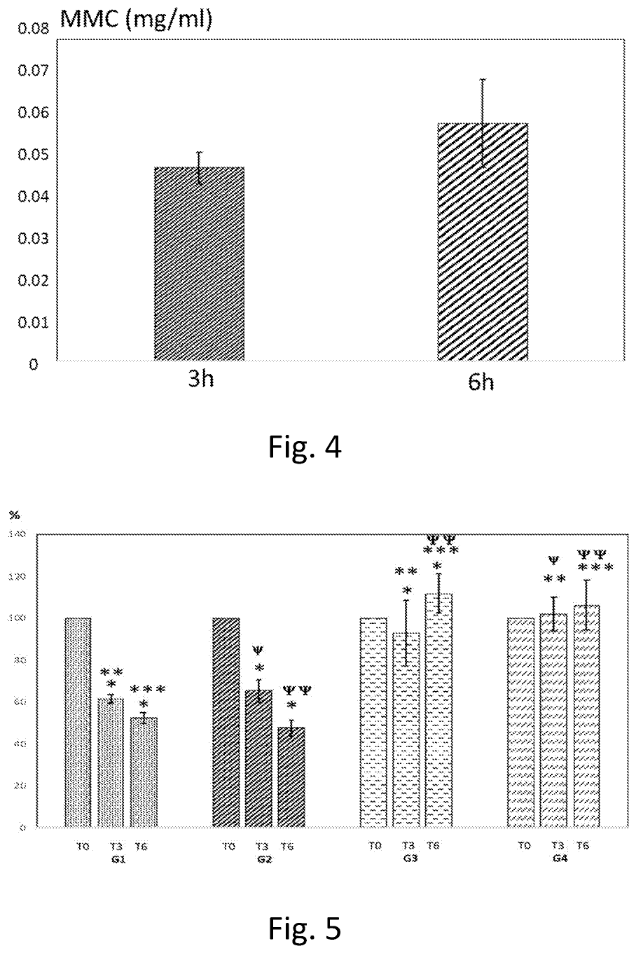

The results of the artificial urine study for the determination of the concentration of MMC released by the BraidStent-SF-MMC at 3 and 6 h are shown in FIG. 4. No significance was found between groups (Student's test). Regarding the percentage of release with respect to the MMC encapsulated in the stent, 81.7% was released at 3 h, and 100% was released at 6 h. The urine study demonstrated the stability of the SF coating in a physiological environment.

Regarding the results of the cell viability % studies according to the determination of absorbance at T3 and T6, the data do not follow a normal distribution.

As for the comparison of the cell viability % between groups over time, two different trends were observed. On the one hand, a trend of G3 and G4 corresponding to the non-MMC groups with BraidStent-SF and T24 cell culture alone (negative control), and on the other hand, the trend of the cultures in which MMC was present in G1 and G2, with a lower cell viability % with respect to the two control groups (FIG. 3).

From the point of view of the tendency over time within each group, we found statistical significance between 3 and 6 h regarding the groups with MMC (G1 and G2), with a decrease in cell viability related to exposure time. In the non-MMC groups, only G3 showed statistical significance over time, increasing its cell viability % (FIG. 3).

In detail, the cell viability of the T24 cells in the presence of G1 and G2 at 3 h were 62.21±2.04% and 65.40±5.26%, respectively. Additionally, at 6 h, it was 52.29±2.42% and 47.66±3.78%, respectively.

Regarding the inter-group comparison of the percentage of cell viability at T24, G1 showed statistical significance at T3 compared to G3 (p=0.013) and G4 (p=0.001), and G2 only showed statistical significance with G4 (p=0.012). At T6, significance was found for the two groups with MMC, G1, and G2 compared to the two negative controls: the G3 (bare BraidStent-SF) culture and G4 (T24 cell culture alone) (p<0.005) (see FIG. 5).

In conclusion, these results show that the intra-ureteral stents coated with the SF matrix and embedded with MMC in its 10 dips (dip-coating technique), allows the controlled release of MMC into the urinary environment at 3 and 6 h. Also, that as there is no statistical difference between G1 and G2, the concentration of MMC released by the intra-ureteral stent according to one or more embodiments of the invention is adequate and comparable to that currently used in patients administered MMC as adjuvant therapy to UTUC.

### Example 2: In Vitro Study MC Release as Adjuvant Therapy in Upper Urothelial Carcinoma

### Materials and Methods

The experimental study was organized into three protocols.

Experimental protocol I. In the first protocol, the aim was to compare two combinations of biodegradable polymers and copolymers for the manufacture of the biodegradable ureteral stent before coating it with SF.

Materials for stent preparation. Three polymers and copolymers were selected for this purpose: Glycomer™ 631 (Biosyn suture by Covidien, Minneapolis, MN, USA), PGA (Safil® Quick suture by B. Braun, Secaucus, NJ, USA), and poly-4-hydroxybutyrate (Monomax® suture by B. Braun Surgical, Barcelona, Spain). All three biomaterials are derived from biocompatible and biodegradable surgical sutures. The search for the right combination of polymers for the biodegradable ureteral stent aimed to produce a stent that degrades within 7-8 weeks in a progressive manner to avoid obstructive degradation in future in vivo studies.

In order to compare the degradation rate, 3-cm long fragments of biodegradable ureteral stents were developed using the following combinations: Group BraidStent-1, a long-term braided stent with Glycomer™ 631 and poly-4-hydroxybutyrate; and group BraidStent-2, a short-term braided stent with Glycomer™ 631 and PGA. The ratio in the composition of the polymers of each stent was always kept constant in their manufacture: Glycomer™ 631 (54%); PGA and poly-4-Hydroxybutyrate (46%).

Five samples of each of the types of stent fragments were developed, giving a total of 10 samples. These were placed in watertight tubes with artificial urine (human synthetic urine, BioIVT, Royston, UK), pH: 6.7 specific gravity 1.008 (FDA registered) for screening studies for high performance liquid chromatography with diode-array detection (HPLC-DAD). To investigate the degradation rate, the stent fragments were dipped in 5 mL of artificial urine (AU) and incubated in an orbital shaker-incubator under mimicked biological conditions (36.5° C. with 5% CO₂, at 90 rpm) until complete stent biodegradation, with daily AU changes. Four follow-ups were performed in each group: TO-start of the study; T1-day of onset of macroscopic degradation; T2—day on which we macroscopically detected that the stent had degraded by 50%; and T3—complete stent degradation. The changes in pH, manifestation of nitrites, weight of the wet stent, and days at which the described changes appeared were assessed. The average thickness of each stent was also determined at baseline (T0).

Experimental protocol II. Once the study corresponding to protocol I had been completed and the most suitable polymer/copolymer combination for the development of the BraidStent® was known, we proceeded to the next step, which consisted of the SF coating of the BraidStent® (the BraidStent-SF group).

Materials for stent preparation and SF coating. SF was obtained from worms as described in Example 1. The resultant 7-8% w/v SF solution was recovered and used for the preparation of the coated BraidStent-SF stents as explained below, adjusting the concentration to 7% w/v before use. The protocol was adapted from the methodology proposed by Rockwood et al. for the manufacture of fibroin tubes by means of a dipping technique using alternate baths of aqueous fibroin and methanol.

The BraidStent-SF group was composed of the same number of stent fragments (five) and followed the same experimental protocol, with the same follow-ups and assessment of the same variables as in protocol I.

Experimental protocol III. Following the evaluation of the BraidStent-SF, it was loaded with two different MMC formulations (BraidStent-SF-MMC1 and BraidStent-SF-MMC2) to assess the release concentration and MMC release time. The BraidStent-SF-MMC1 and 2 groups were composed of the same number of stent fragments, 5 per group, and followed the same experimental protocol and follow-ups as in protocols I and II.

Materials for stent preparation and SF and MMC coating. For the BraidStent-SF-MMC1 group, the coating of the stents used powdered 10 mg MMC to produce the intravenous injectable solution commonly used in the medical field (INIBSA, Barcelona, Spain); the vials contained sodium chloride as an excipient (9.5 mg of sodium per 1 mg of MMC). For this, a 7% w/v fibroin solution containing 5 mg/mL of MMC was prepared by dissolving the drug for 30 min under orbital agitation at 120 rpm. At the same time, the same protocol was performed using absolute methanol, dissolving MMC at the same concentration and for the same period of time. These two starting solutions were used to alternately bathe the stent fragments, by dipping for 5 s in the first solution (of aqueous fibroin) and then in the second (containing methanol), for the same time, and letting them dry slightly for 1 min before repeating the procedure. These coating cycles were carried out in this BraidStent-SF-MMC1 group 10 times. The containers used for this purpose were 5 mL glass tubes.

For the BraidStent-SF-MMC2 group, the concentration employed for both the fibroin and methanol solutions was 10 mg/mL, and 10 dip coating cycles were performed. In this group, 70 mg of pure MMC (Mitomycin CRS, Sigma-Aldrich, Steinheim am Albuch, Germany) was used, without any excipient.

The stents were completely stable in room air. However, the MMC coating needed special care, i.e., it must be packaged in a lightproof for preservation and transportation to ensure its physical and chemical stability. MMC shows some light sensitivity, so reasonable steps to minimize light exposure should be taken.

MMC assessment. MMC concentrations were assessed every six hours until there was no analytical evidence of its detection. Urine from each follow-up was replaced and analysed. MMC was released from the SF matrix due to solubility events of the SF matrix in the urinary environment. The permeability and release kinetics of the MMC depends on the SF coating and relates to the percentage of the beta-sheet structure. Increasing the crystallinity (methanol immersion) of the SF beta sheet decreases the release rate and increases the release duration.

The HPLC-DAD method was an isocratic method with a mobile phase of acetonitrile: ultrapure water 80:20 (v/v) at a flow rate of 1 mL/min, and separation was performed on a LUNA C18 250 mm, 4.6 mm, 5 μm column at 30° C. temperature. A total of 10 μL of sample was injected. The MMC was detected with a diode array detector (DAD) at 365 nm (1260 Infinity II Prime LC System, Agilent Technologies, Santa Clara, CA, USA).

Statistical analysis. Statistical analysis was performed with the SPSS 25.0 program for Windows (IBM, Armonk, NY, USA). The variables studied were pH, weight of the stents in g, degradation rate expressed in days, stent width in mm, and artificial urine concentration of MMC in mg/L. Variables are shown as their mean±standard deviation. The normality of the data was analysed using the Shapiro-Wilk test. For data that followed a normal distribution, the intra-group and intra-phase distributions were analysed using a one-factor ANOVA. Post-hoc analysis was performed using Tukey's HSD (honestly significant difference) test.

For variables in which the data were not normally distributed, the comparison between groups in each phase was carried out using the Kruskal-Wallis test. In the event of statistical significance, the corresponding post-hoc pairwise comparison was carried out.

The trends of the variables throughout the follow-ups concerning the effect of the factors time and group, were analysed by means of a General Linear Model (GLM) Repeated Measures. Again, the post-hoc analysis was performed using Tukey's HSD. In certain cases, to evaluate the trend of each group along time, a Friedman test or a Wilcoxon test was performed, depending on the number of follow-ups included in the analysis. In addition, the concentration of Mitomycin C released by the two groups, BraidStent-SF-MMC1 and BraidStent-SF-MMC2, was analysed either via a t-test for independent samples or a Mann-Whitney test, depending on the normality of data. Confidence intervals were set at 95% and significance was determined by p-values less than 0.05.

### Results

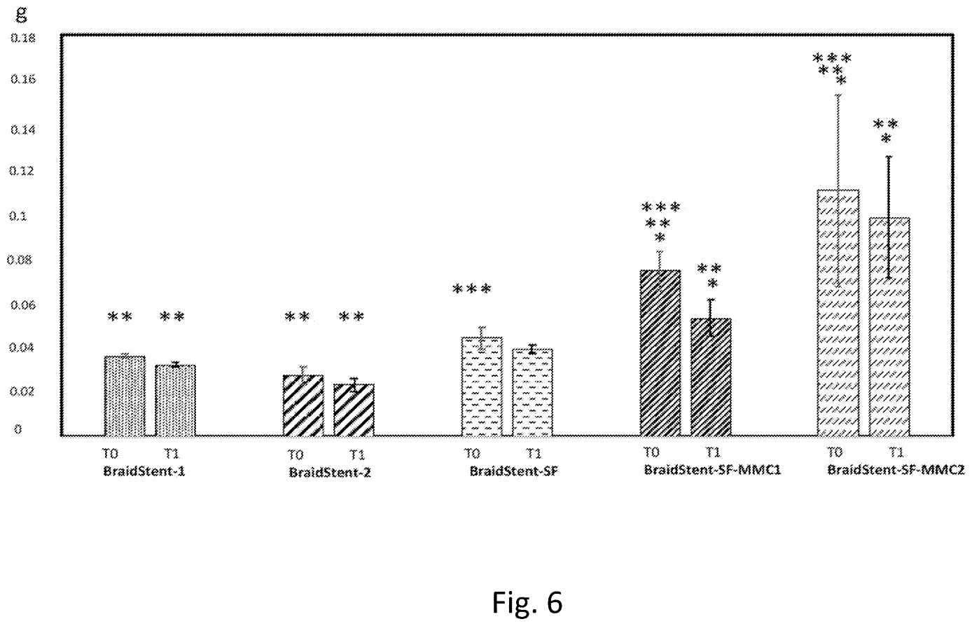

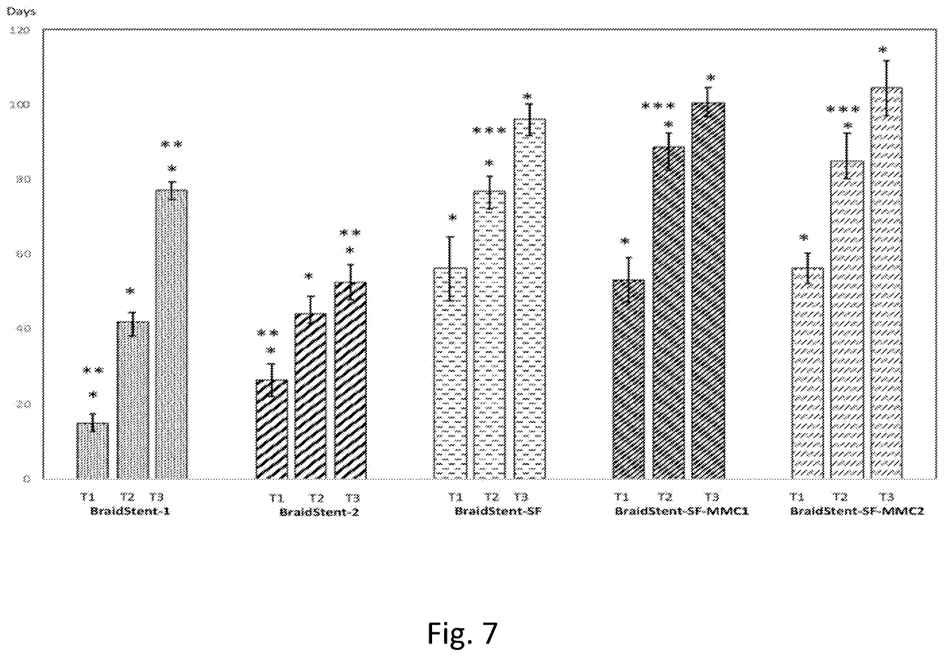

Protocol I. We found significant differences between the two groups for the variable ‘stent weight’ at T0 and T1. The combined weight of polymers and copolymers in BraidStent-1 was significantly higher than in BraidStent-2. BraidStent-1 showed a weight loss of 11.11% and BraidStent-2 showed a weight loss of 14.81% between the baseline study and the macroscopic onset of degradation (FIG. 6). The duration of degradation variable shows a statistically significant difference between both groups at T1 and T3, but not at T2 (FIG. 7).

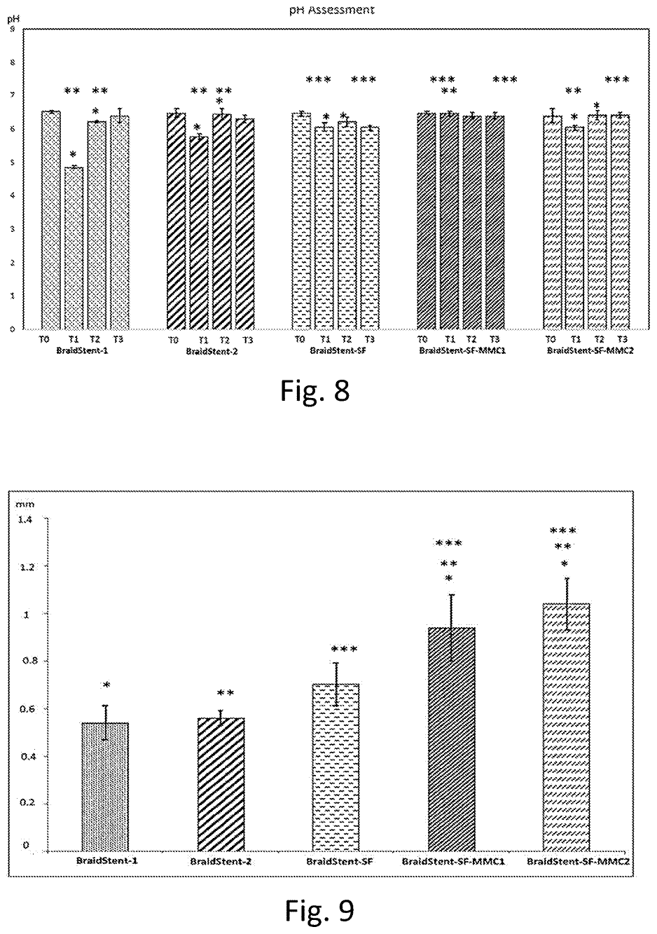

BraidStent-2 started degrading later, but between T2 and T3, there was an acceleration of the hydrolysis process, resulting in a shorter time to complete stent degradation. BraidStent-2 fit the criterion of degradation in the first 7-8 weeks (FIG. 5). The difference in the pH of the medium in protocol I showed statistical significance at T1 and T2. The stent degradation metabolites of BraidStent-1 and BraidStent-2 caused overt acidification of the medium, which was more marked with BraidStent-1. At T2 and T3, the pH returned nearly to basal levels. Using Friedman's statistical test, we assessed the trend over the different phases and whether the changes in pH within each group were uniform (FIG. 8). In both groups, the pH trend was not uniform and showed significance throughout the different phases (BraidStent-1 group p=0.006 and BraidStent-2 group p=0.009). None of the samples were positive for nitrites on urinalysis.

Protocol II. For this protocol, the BraidStent-2 group was selected as it met the inclusion criteria of complete degradation before 8 weeks. The BraidStent-SF group used the same combination of polymers and copolymers as in the BraidStent-2 group, dip-coated with SF. The characterization results of the comparative study between BraidStent-2 and BraidStent-SF SRB coated show, after evaluation with fluorescence microscopy, that the SF coating of the stent is uniform.

The BraidStent-SF group did not show statistically significant changes between TO and T1, with a decrease in the weight of 11.36% between the two phases (FIGS. 4 and 8). With regard to degradation time, the addition of the SF coating led to significant differences compared to the BraidStent groups, significantly increasing the degradation time compared to the stent without the SF coating at T1-T2-T3 (Kruskal-Wallis H test, p=0.002). The Friedman test determined that the trend in pH between the study phases showed statistical significance (p=0.016). As in groups BraidStent-1 and -2, there was acidification of the urinary medium at the beginning of degradation, which subsequently recovered to basal levels at T3 (see FIG. 8). None of the samples were positive for nitrites on urinalysis, ruling out bacterial contamination.

Protocol III. Following the addition of the two MMC formulations to the stent, the weight showed statistically significant changes between the BraidStent-SF-MMC1 and the BraidStent-SF-MMC2 groups, with a greater weight in the latter group at TO and T1. Both groups showed statistically significant differences compared to the SF-coated stent without MMC; thus, the addition of MMC significantly increased the weight of the stent (see FIG. 6). Moreover, stent thickness showed statistically significant differences between the BraidStent-SF group and the two groups coated with MMC (see FIG. 9). There was no statistically significant difference between the BraidStent-SF-MMC1 and the BraidStent-SF-MMC2 groups with regard to the manifestation of the different degradation phases, however, when both groups were compared with BraidStent-SF, statistical significance was found at T2, with this latter group showing a much faster degradation at this follow-up (see FIG. 7). The BraidStent-SF-MMC1 group, in contrast to BraidStent-SF-MMC2, shows a uniform trend in its variations throughout the urinary pH study (Friedman's test, p=0.357) (see FIG. 8). Both stent groups underwent the same number of coating cycles, 10, although the initial concentration of MMC in BraidStent-SF-MMC1 was 10 mg compared to 70 mg in BraidStent-SF-MMC2. In addition, the joint dilution of SF and MMC was 5 mg/mL and 10 mg/mL, respectively. The kinetics of MMC in both groups was fully released within the first 12 h, with no analytical evidence of MMC afterward. We found statistically significant differences in the concentration of MMC released at 12 h between the groups, with a higher urine concentration of MMC in the BraidStent-SF-MMC2 group (Table 1). Statistically significant differences were also found within each group between 6 and 12 h. None of the samples were positive for nitrites on urinalysis.

TABLE 1
Mitomycin C concentration release rate (mg/L). MMC release is
assessed every 6 h in artificial urine in BraidStent-SF-MMC1
and BraidStent-SF-MMC2 stents, until it is not detected by HPLC-
DAD. The variable mg/L MMC at 6 h follows a normal distribution
but not at 12 h. A Student's t-test for independent samples
was performed in order to determine the differences at 6 h between
groups. As for the study at 12 h, a Mann-Whitney test was performed.
The trend in MMC concentration over the 6 and 12 h, between
groups and within each group, was analysed using a GLM Repeated
measures. Superscripts refer to statistical significance between
the values, namely inter- or intra-group. a-b-c (p < 0.01).
Therefore, the addition of mitomycin C to the coating of silk
fibroin biodegradable intra-ureteral stents according to the
present invention enables the <intracavitary instillation
in the upper urinary tract.

| mg/L | 6 h | 12 h | 18 h | 24 h |
|---|---|---|---|---|
| BraidStent-SF-MMC1 | 10.98 ± 3.73 ^{a} | 2.33 ± 3.05 ^{ac} | 0 | 0 |
| BraidStent-SF-MMC2 | 7.67 ± 1.39 ^{b} | 56.08 ± 4.76 ^{bc} | 0 | 0 |

### Example 3: Animal Model Study MC Release as Adjuvant Therapy in Upper Urothelial Carcinoma

### Materials and Methods

### Animal Model Study: BraidStent-SF-MMC Stent

Fourteen healthy large white breed female pigs were used in this study. The experimental protocol was approved by our institutional animal Care Committee (Directive 2010/63/EU on the protection of animals used for scientific purposes; Reference: 011/18).

### Phase 1: Baseline Study-Solitary Kidney Animal Model

After mandatory quarantine, the animals were evaluated by hematologic blood chemistry and urinalysis to assess health status (Table I). next, a nephrosonographic study was performed to assess pyelocaliceal dilatation. 14, 15 Simulated voiding cystourethrography (SVCUG) was carried out to assess vesicoureteral reflux (VUR) at baseline and all follow-up assessments. 16 Finally, excretory urography was performed to assess UUT morphology. The inclusion criteria were: absence of anatomical abnormalities and negative urine culture. The parameters were within the reference range for swine species. Afterwards, a laparoscopic left RNU was performed. The decision to include solitary kidney animal models allows us to accurately quantify the concentration of MMC released and to evaluate only the adverse effects and alterations in blood biochemistry and urinalysis related to BraidStent-SF-MMC stenting, with no contralateral kidney compensatory response.

To control urinary pH, one week prior to stenting, NaHCo₃ was given orally at a dose of 2×4 g/day until the end of the study. In previous BraidStent-SF-MMC in vitro studies, the SF has been shown to behave unstably at urinary pH<7.12 Therefore, all animals were strictly controlled to alkalinize the pH and to ensure that their pH was ≥7 before stenting.

### Phase 2: Stenting—Immediate Post-Stenting Follow-Up (0-48 h)

Six weeks after RNU, the health status of the animal and the urinary tract were assessed by the following diagnostic techniques, which were used in all subsequent follow-up assessments: nephrosonographic study, SVCUG, excretory urography, retrograde ureteropyelography, ureteroscopy, hematology blood chemistry and urinalysis, followed by BraidStent-SF-MMC placement. The inclusion criteria in this phase were the same as in the previous phase, with the addition that, prior to ureteral stenting, all animals must show a pH≥7.

The stent employed was a biodegradable and intraurethral design, BraidStent®, previously validated by our research group. 12, 16 The BraidStent® was placed using the standard transurethral approach by sliding over a guide under fluoroscopy control. The 3Fr BraidStent® was designed to avoid disrupt the ureterovesical junction antireflux mechanism to prevent bladder trigone irritation and VUR, with a short length of 14 cm (see FIG. 3). For the BraidStent-SF-MMC manufacturing, a 7% w/v fibroin solution containing 10 mg/ml of pure MMC was prepared by dissolving the drug for 30 min under orbital agitation at 120 rpm. These dip coating cycles were carried out 10 times. The stents were completely stable in room air with lightproof packaging.

After transurethral stenting, a 24 Fr bladder catheter was placed and remained occluded for 30 h. Every 6 hours, the bladder urine was emptied for urinalysis, as well as for high-performance liquid chromatography diode array detector (HPLC-DAD) determination of MMC urine concentration. at 48 h, nephrosonography and a urine and blood sample were collected. Systemic toxicity was evaluated by clinical examination, hematology and blood chemistry analysis and MMC plasma concentration during all follow-up studies.

### Phase 3: Follow-Up

Follow-up assessments were done weekly with the above-mentioned diagnostic procedures until complete stent degradation. The follow-up allowed us to assess the development of obstructive uropathy, drainage of the upper urinary tract, the onset of urothelial inflammation, the macroscopic condition of the BraidStent-SF-MMC and its degradation, together with its correct location, as well as alterations in laboratory parameters and urine contamination. Complications arising at the UUT were also evaluated. a complication score was developed: 0=no complications; 1=appearance of complications.

### Phase 4: End-Study Assessment

The final assessment was carried out when the weekly follow-up confirmed complete stent degradation. The experimental study was finished by removing the urinary tract end bloc for macroscopic and pathological study. histological slices were obtained from the ureteropelvic junction and kidney.

Success was strictly defined as the following: stent degradation between 6-7 weeks; hydronephrosis SFU Score≤1; no ureteral stricture; serum creatinine≤2.30 mg/dL. If these four items were met in the final assessment, the animal was graded as successful. if it failed one of the items, it was graded as disappointing. if it failed more than one, it was graded as unsuccessful.

### Statistical Analysis

Statistical analysis was performed with graphpad prism 8.3.0 software (dotmatics, UK). The sample size was 14 animals (0.05 level of significance; 90% statistical power). The normality of the data was analysed using the Shapiro-Wilk Test. For data that followed a normal distribution, their follow-up trending was analysed using a repeated measures anova. post-hoc analysis was performed using Tukey's Multiple Comparisons Test. For variables that did not follow a normal distribution, the comparison in each phase was carried out using the Friedman test, while dunn's multiple comparisons test was chosen for pairwise comparisons. The trend of categorial and dichotomous variables was analysed via the x2 Test. To assess the potential role of urine pH as risk factor for both success and complication rates, a mixed effects logistic regression model was carried out.

### Results

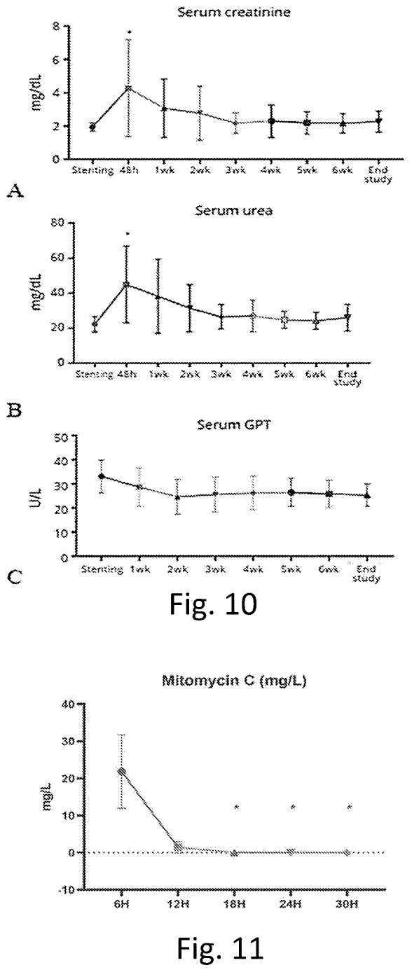

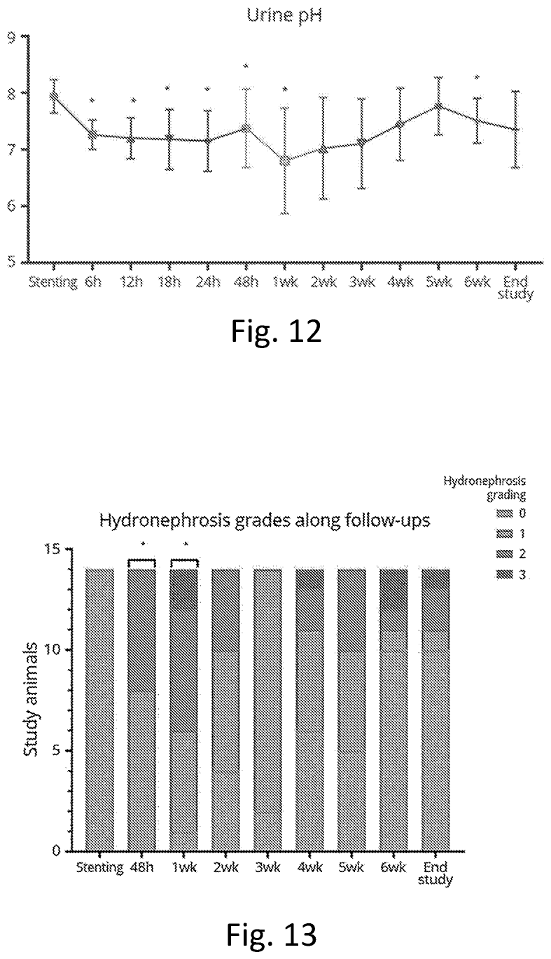

Phase 1: no animals showed any alterations; negative urine cultures and serum values were within swine reference intervals (see FIG. 10).
Phase 2: study animals that fulfilled the inclusion criteria at the time of stenting had a pH≥7.0. The BraidStent-SF-MMC was successfully placed in all 14 animals. MMC release took place mainly between 0-6 h in 93.2% of animals, with statistical significance from 18 h onwards (see FIG. 11). Significance was found with respect to urinary pH between stenting and all follow-up assessments, up to 48 h. of note, the only three animals classified as unsuccessful showed a mean pH<7.0 between 0-48 h (FIG. 12). There was a significant increase between stenting and the 48-h follow-up regarding the hydronephrosis score (FIG. 13). all study animals showed an increase in the degree of hydronephrosis. on the other hand, serum urea and creatinine mean values showed a significant increase at 48 h, above the normal range in porcine species (FIG. 2a, B). Serum glutamic-pyruvic transaminase (GPT) remained unchanged, which suggested the absence of acute hepatotoxicity (FIG. 2C). Serum MMC values were found lower than HPLC-DAD detection limit (<0.1 mg/L).
Phase 3: regarding the urinalysis results, the pH dropped significantly below 7.0 in the first week, gradually recovering to baseline pH values from the second week onwards. From the first to the third week, the pH in the animals rated as unsuccessful was below 7.0, unlike the rest of the animals. The adjusted odds ratio (aOR) showed a significant association between high pH and success rate (aOR=1.648 103, 95% Ci: 1.646 103-1.650 103, p<0.001). an association was also observed between lower pH values and higher complication rates (aOR=0.53, 95% CI: 0.27-1.01, P=0.0524), although this was not significant. Finally, the aOR of coating fragments according to pH values showed a non-significant relationship between lower pH and the risk of developing BUS coating fragments (aOR=0.45, 95% CI: 0.14-1.44, P=0.159). The lack of statistical significance in the latter two aOR, may be due to the low representation of the variables' complications and coating fragments, in this 14-animal study. The rate of positive nitrite during follow-up assessments was 6.08%, with no statistical significance. The degree of hydronephrosis increased significantly one week after stenting, decreasing significantly over the remaining follow-up assessments, neither serum creatinine, urea, or GPT showed significance over follow-up versus stenting values.

Assessment of BraidStent-SF-MMC-related complications found ureteral strictures only in the three animals classified as unsuccessful, which appeared between the fourth and sixth week. On the other hand, four percutaneous nephrostomies (PCN) were required during the first week due to the unscheduled depolymerization of the SF matrix. The presence of obstructive coating fragments was detected by ureteroscopy and ureter pyelography in the first to third weeks at 28.5% and 7.1%, respectively. Samples of obstructing fragments retrieved by ureteroscopy were analysed by infrared spectroscopy and it was confirmed that they were exclusively SF.

Phase 4: 93% of the stents were completely degraded at 7 weeks and the remaining at 6 weeks without obstructive fragments. The mean complication rate in the final assessment was 25.7% (Table II). Urinary pH values were completely restored. Serum creatinine, urea, and GPT were within the reference intervals. The final treatment success rate was 67.5%. No major pathological changes were observed. The most common findings were mild multifocal leukocyte infiltrates in the kidneys, as well as thinning of the urothelial cell layers, and erosion and mild and diffuse lymphoplasmacytic infiltrate in the ureteral lamina propria in some animals.

In conclusion, the biodegradable intra-ureteral stent, according to the present invention allows for controlled and well-tolerated release of MMC into the UUT in a porcine model. MMC release from a SF coating of a BUS could be a compelling approach for adjuvant chemotherapy after endoscopic upper tract urothelial carcinomas management.

1. Intra-ureteral stent that comprises:
a. a jay shaped hollow tube (1) with biomechanical memory on its proximal end, which serves to insert and anchor the device in an ureter;
b. a main body (2) made of a plurality of biodegradable intertwined threads (4) which facilitate urine drainage and;
c. a distal end (3), configured to remain inside of the ureter;
wherein the main body (2) connects the jay shaped hollow tube (1) with the distal end (3),
characterised in that at least two of the intertwined threads (4) comprise different materials each with different degradation rates, and in that the main body (2) is coated with a polymeric matrix (5), the polymeric matrix (5) comprising at least one active ingredient.

2. The intra-ureteral stent of claim 1, wherein the polymeric matrix (5) comprises silk-fibroin, which in turn comprises the active ingredient, preferably embedded within the fibroin.

3. The intra-ureteral stent according to any one of the previous claims, wherein the polymeric matrix (5) comprises different active ingredients concentrations at different depths.

4. The intra-ureteral stent according to any one of the previous claims, wherein the at least one active ingredient is selected from the group consisting of: chemotherapy agents, immunotherapy agents, antibiotic agents, and/or anti-inflammatory agents.

5. The intra-ureteral stent according to claim 4, wherein the chemotherapy agent is selected from any one of the following list consisting of: mitomycin, epirubicin, paclitaxel, docetaxel, valrubicin, and/or doxorubicin.

6. The intra-ureteral stent according to any one of the previous claims, wherein the polymeric matrix (5) is configured to biodegrade and elute the at least one active ingredient in a controlled pace through hydrolysis.

7. The intra-ureteral stent according to any one of the previous claims, wherein the biodegradable intertwined threads (4) comprise a polymer and/or copolymer.

8. The intra-ureteral stent according to claim 7, wherein the polymer and/or copolymer comprise any of the materials selected from the following list consisting of: glycomer-631, polyglactin, polycaprolate, polylactic acid, polyglycolic acid, alginate, gellen, poly-4-hydroxybutyrate.

9. The intra-ureteral stent according to any one of the previous claims, wherein one or more of the at least one active ingredient is an antitumoral agent, for use in treatment of urothelial cancer.

10. The intra-ureteral stent according to any one of the previous claims, wherein one or more of the at least one active ingredient is an antitumoral agent for use as an adjuvant therapy, for use in treatment of urothelial cancer.

11. The intra-ureteral stent according to any one of the previous claims, for use in treatment of urothelial carcinoma.

12. The intra-ureteral stent according to any one of the previous claims, for use in treatment of urothelial neoplasm associated to bladder cancer.

13. The intra-ureteral stent according to any one of the previous claims, wherein one or more of the at least one active ingredient is an analgesic and/or anti-inflammatory agent, for use in treatment of urinary tract inflammations.

14. The intra-ureteral stent according to any one of the previous claims, wherein one or more of the at least one active ingredient is an antibiotic agent, for use in the treatment of urinary tract infections and/or bacteriuria.
